(19) United States
(12) Patent Application Publication
(10) Pub. No.: US 2021/0022842 A1
(43) Pub. Date: Jan. 28, 2021

(54) SELF-AFFIXING MEDICAL DEVICES AND ADDITIVE MANUFACTURE OF SAME

(71) Applicant: Poly-Med, Inc., Anderson, SC (US)

(72) Inventors: Michael Scott Taylor, Anderson, SC (US); Brian Gaerke, Travelers Rest, SC (US); Seth Dylan McCullen, Greenville, SC (US); Ryan Andrew Borem, Anderson, SC (US)

(21) Appl. No.: 16/936,209

(22) Filed: Jul. 22, 2020

Related U.S. Application Data
(60) Provisional application No. 62/877,152, filed on Jul. 22, 2019.

(51) Int. Cl. A61F 2/00; B33Y 80/00; B33Y 10/00; B33Y 50/02; B29C 64/393

(52) U.S. Cl. CPC A61F 2/0063; B33Y 80/00; B33Y 10/00; B33Y 50/02; B29L 2031/753; A61F 2002/0068; A61F 2210/0004; A61F 2240/004; A61F 2220/0008; B29C 64/393

(57) ABSTRACT

Disclosed herein are medical devices comprising biocompatible substrates and one or more fixation elements, and methods for making and using the same.

### CROSS-REFERENCE TO RELATED APPLICATION

This application claims the benefit under 35 U.S.C. § 119(e) of U.S. Provisional Patent Application No. 62/877,152, filed Jul. 22, 2019, which application is incorporated herein by reference in its entirety for all purposes.

### TECHNICAL FIELD

The present disclosure relates generally to implantable self-affixing medical devices comprising at least one fixation element and to methods for forming such devices.

### BACKGROUND

Treatment of parietal insufficiencies generally require the use of reinforcing medical devices such as surgical meshes. Surgical meshes may be used during both laparoscopic and open surgery for repair of many types of defects and injuries. Methods for hernia and eventration repairs, e.g., inguinal and abdominal, and reconstructions for soft tissue and muscle wall damage often employ meshes for mechanical support of the injured tissue. Meshes may be used to provide support to surrounding tissue, as well as to supplement standard suturing.

During hernia repair, the mesh may be placed over the entirety of damaged tissue and some of the healthy tissue surrounding the defect. The mesh can be held in place by a fixation device that attaches the mesh to the surrounding tissue. A variety of different fixation devices may be used to anchor the mesh into the tissue. For example, a needled suture may be passed through or around the tissue near the defect to hold the mesh in a position which spans the injured tissue. In other instances, staples, tacks, clips and pins are known to be passed through or around the tissue near the defect to anchor the implant in a position which spans the injured tissue.

Unfortunately, the use of such fixation devices may increase the patient's discomfort and, in certain instances, may weaken the tissue to which the fixation devices are attached. Certain techniques involve placing a mesh against the repair site without the addition of a fixation device. For example, in some instances the mesh may be simply positioned within the abdomen allowing the pressure of the peritoneum to hold the mesh against the posterior side of the abdominal wall. However, fixation of the mesh may be helpful in order to avoid folding, shrinkage, and migration of the mesh.

Although, methods that require the use of fixation devices have been proven effective in anchoring an implant, such as a mesh into the tissue, penetration of the tissue by such devices inflicts additional trauma to the damaged tissue or the tissue near the defect and requires additional time for healing. Thus, implantable devices which do not require the use of sutures, staples, tacks, pins, and/or clips is desirable in order to further limit the amount of trauma to healthy tissue surrounding the wound and caused by the fixation devices.

What is needed are medical devices comprised of biocompatible substrates that can at least initially self-affix to a subject's tissue due to the presence of fixation elements, such as barbs or nibs, located on a surface of the substrate, such as at fiber contact points of the substrate, that are easily manufactured, and optionally, all or a portion of the fixation elements, such as barbs or nibs, and/or biocompatible substrate are bioresorbable.

### SUMMARY

The present disclosure comprises methods and compositions comprising a medical device made of a material having contacting fibers, such as a mesh or electrospun material, wherein 3D-printed fixation elements, such as barbs or nibs, are printed onto the site of at least two fibers contacting each other or the site where a portion of a fiber contacts another portion of the fiber, herein “fiber contacting site”. The present disclosure comprises methods and compositions comprising a medical device made of a material having defined, identified or determined sites for attaching fixation elements by 3-D printing. For example, a 3D-printed fixation element, such as a barb or a nib, is printed onto one site, or a plurality of fixation elements are each printed onto one of a plurality of identified sites of a surface of the medical device. An identified site is a site chosen on a surface of a medical device onto which at least one attachment element is 3D-printed using an additive manufacturing device. Generally, the medical device is implantable, which may include implantation within a subject's body and implantation on a surface of a subject's body, e.g., contacting external skin or the surface of an internally located organ or tissue.

In an aspect, a medical device includes a biocompatible substrate having a surface comprising at least one fixation element, such as a barb or nib. The at least one fixation element may protrude, for example perpendicularly, from the surface of the biocompatible substrate. In an aspect, a plurality of fixation elements may be positioned along any portion of the surface of the biocompatible substrate at identified sites or at locations where at least one fiber portion overlaps, underlaps or contacts at least one other fiber portion. As used herein, “fiber contacting site” may comprise one separate fiber contacting a second separate fiber or may comprise a first portion of a fiber contacting a second portion of the same fiber. As used herein, fixation element, includes, but is not limited to, one or more barbs, nibs or spiked naps, and is a member formed, by additive manufacturing methods, which includes a linear portion of which one end is affixed to a substrate, such as a biocompatible substrate, and the opposite end may or may not terminate in a shaped element.

In an aspect, a tissue-gripping implantable medical device disclosed herein may include a biocompatible substrate having a surface comprising one or more fixation elements. The fixation elements may protrude perpendicularly, or in any angled direction, from the surface of the biocompatible substrate. In an aspect, the one or more fixation elements may be positioned along any portion of the surface of the biocompatible substrate, for example, wherein a fixation element is positioned at a site where at least one fiber portion contacts or overlaps another fiber portion, termed herein as a fiber contacting site, or wherein a fixation element is positioned at an identified site.

Methods of forming such devices are disclosed. In an aspect, methods of forming a tissue-gripping implantable medical device are disclosed, comprising, using an additive manufacturing device (e.g., a 3D-printing device, wherein FDM, SLA or other 3-D printing devices and methods are employed) to affix at least one fixation element to a biocompatible substrate at an identified site or a fiber contacting site, e.g., locations of the substrate wherein at least one fiber portion contacts a second fiber portion (referred to herein as a fiber contacting site) to form a biocompatible substrate comprising at least one fixation element. Generally, a plurality of individual fixation elements are printed onto a plurality of individual fiber contacting sites to form a plurality of fixation elements on a surface of a biocompatible substrate. Generally, a plurality of individual fixation elements are printed onto a plurality of identified sites to form a plurality of fixation elements on a surface of a biocompatible substrate. Such methods produce tissue-gripping implantable medical devices which comprise 3D-printed fixation elements, such as barbs.

### BRIEF DESCRIPTION OF THE DRAWINGS

FIG. 1A shows an illustration of exemplary fiber contacting sites of a mesh medical device.

FIG. 1B shows an illustration of a close-up view of an exemplary fiber contacting site

FIG. 2 shows an exemplary tissue-gripping medical device with fixation elements printed on the periphery.

FIG. 3 shows an exemplary fixation element.

It should be understood that aspects of the disclosure are described herein with reference to the figures, which show illustrative embodiments in accordance with aspects of the disclosure. The illustrative embodiments described herein are not necessarily intended to show all aspects of the disclosure, but rather are used to describe a few illustrative embodiments. Thus, aspects of the disclosure are not intended to be construed narrowly in view of the illustrative embodiments. It should be appreciated, then, that the various concepts and embodiments discussed herein may be implemented in any of numerous ways, as the disclosed concepts and embodiments are not limited to any particular manner of implementation. In addition, it should be understood that aspects of the disclosure may be used alone or in any suitable combination with other aspects of the disclosure.

### DETAILED DESCRIPTION

Disclosed herein are methods, devices and compositions comprising a biocompatible substrate comprising at least one fixation element, wherein the fixation element is located at an identified site or at a fiber contacting site of the biocompatible substrate. As used herein, a fiber contacting site is a site where at least one portion of a fiber overlaps or contacts a second portion of the same fiber, or where at least a first fiber contacts at least a second fiber. As used herein, an identified site is a site on a surface of a medical device that has been selected (“identified”) as a site onto which a fixation element is to be printed. Identified sites may be determined on surfaces of medical devices that do not comprise fibers or medical devices that do not comprise overlapping fibers. The present disclosure comprises implantable medical devices, comprising one or more fixation elements, which have tissue-gripping capabilities. In an aspect, an implantable medical device includes at least one fixation element that is capable of snagging or attaching to tissue, or embedding or penetrating into tissue.

Disclosed implantable medical devices include a biocompatible substrate having a surface to which at least a fixation element may be positioned. In an aspect, biocompatible substrates are often planar in configuration, however, any two-dimensional or three dimensional shapes suitable for implantation may be used. Some examples of suitable biocompatible substrates include films, foams, meshes, buttresses, patches, tapes, pledgets, occlusion devices, and the like. In an aspect, a biocompatible substrate is a surgical mesh.

Any biocompatible material may be used to form the biocompatible substrates and/or the fixation elements described herein. For example, the substrate may be made from natural, synthetic, bioabsorbable or non-bioabsorbable materials. It should of course be understood that any combination of natural, synthetic, bioabsorbable and non-bioabsorbable materials may be used to form the substrates or fixation elements described herein. The term “bioabsorbable” as used herein is defined to include both biodegradable and bioabsorbable materials. By bioabsorbable, it is meant that the materials decompose, or lose structural integrity under body conditions (e.g. enzymatic degradation or hydrolysis) or are broken down (physically or chemically) under physiologic conditions in the body such that the degradation products are excretable or absorbable by the body.

### Materials for Substrates and Fixation Elements

Substrates and/or fixation elements may be formed from bioresorbable or bioabsorble polymers, including but not limited to, poly(alpha-hydroxy acid) polymers and copolymers, such as polymers and copolymers of glycolide including polyglycolide (PGA), poly(glycolide-co-lactide) (PGLA), and poly (glycolide-co-trimethylene carbonate(PGA/TMC; polymers and copolymers of polylactide (PLA) including poly-L-lactide (PLLA), poly-D-lactide (PDLA), poly-DL-lactide (PDLLA), poly(lactide-co-tetramethylene glycolide), poly(lactide-co-trimethylene carbonate), poly(lactide-co-delta-valerolactone), poly(lactide-co-epsilon-caprolactone), poly(glycine-co-DL-lactide) and poly(lactide-co-ethylene oxide); polymers and copolymers of caprolactone or ϵ-caprolactone; polydioxanone polymers such as asymmetrically 3,6-substituted poly-1,4-dioxane-2,5-diones; poly(beta-hydroxybutyrate) (PHBA) and copolymers of the same such as poly(beta-hydroxybutyrate-co-beta-hydroxyvalerate); polygluconate; poly(beta-hydroxypropionate) (PHPA); poly(beta-dioxanone)(PDS); poly(delta-valerolactone); poly(ϵ-caprolactone); methylmethacrylate-N-vinylpyrrolidone copolymers; polyester amides; polyesters of oxalic acid; polydihydropyranes; poly(alkyl-2-cyanoacrylate); polyvinyl alcohol (PVA); polypeptides; poly(beta-maleic acid)(PMLA); poly(beta-alkanoic acid); poly(ethylene oxide) (PEO); polyanhydrides, polyphosphoester, and chitin polymers. Other useful bioresorbable polymers or copolymers comprise monomers, polymers and copolymers taught in PCT Application Serial No. PCT/US2020/021499, herein incorporated in its entirety for its teaching of monomers, polymers and copolymers.

In an aspect, a disclosed polymer is a polyester. For example, a polymer may be a polyester selected from poly(a-hydroxy acid) homopolymers, poly(alpha-hydroxy acid) copolymers and blends thereof. In addition or alternatively, the polyester may be selected from polyglycolide, poly-L-lactide, poly-D-lactide, poly-DL-lactide, and blends thereof. The polyester may be selected from polymers and copolymers of polylactide (PLA), including poly-L-lactide (PLLA), poly-D-lactide (PDLA), poly-DL-lactide (PDLLA).

In an aspect, a polymer is semicrystalline, or is capable of being formed into fibers, or is both semicrystalline and fiber-forming. A fast-degrading polymer may comprise glycolide as the, or one of the, monomer(s) used to form the polymer. Para-dioxane (PDO) is another suitable monomer for forming fast-degrading polymers, where the corresponding homopolymer is known as poly(PDO). Poly(PDO) typically degrades more slowly that glycolide-based polymer, so in order to prepare a very fast degrading polymer, the monomer feed is preferably rich in glycolide.

In an aspect, a disclosed polymer has a polyaxial structure. In an aspect, a polymer is linear. The polyaxial structure may be a part of the polymer, for example, it may be present in a block of a block copolymer. Another option is for the polymer is a segmented polyaxial that is semicrystalline and fiber-forming, and may be glycolide-based for fast degradation. In an aspect, linear copolymers may be comprises of either or both of: diblock, triblock, pentablock, wherein the central block is amorphous and the other blocks are semicrystalline. A pentablock polymer, may comprise (polyethylene glycol) PEG as a central block with amorphous segments connected to the outer crystalline segments (forming a symmetrical pentablock polymer that is a polyether-ester). In an aspect, linear block copolymers may also comprise semicrystalline blocks, with no amorphous blocks, resulting in polymers that can be oriented after fiber formation to create alternating patterns of different crystalline structure and percentage in the fiber, such that there is slight differences in degradation profile of the alternating blocks forming the fiber (as a fiber is oriented, horizontal strips of crystalline regions form and align the blocks comprising the polymer chain). Alternatively, unblocked linear copolymers can be substituted.

Other bioabsorbable materials include: polysaccharides, such as alginate, dextran, chitin, hyaluronic acid, cellulose, collagen, gelatin, fucans, glycosaminoglycans, and chemical derivatives thereof (substitutions and/or additions of chemical groups, for example, alkyl, alkylene, hydroxylations, oxidations, and other modifications routinely made by those skilled in the art); and proteins, such as albumin, casein, zein, silk, and copolymers and blends thereof, alone or in combination with synthetic polymers.

Modified polymers include, but are not limited to, cellulose derivatives, such as alkyl celluloses, hydroxyalkyl celluloses, cellulose ethers, cellulose esters, nitrocelluloses, and chitosan. Examples of suitable cellulose derivatives include methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, and cellulose sulfate sodium salt, referred to herein as “celluloses.”

Other bioabsorbable polymers include polyhydroxy acids prepared from lactone monomers, such as glycolide, lactide, caprolactone, ϵ-caprolactone, valerolactone, and δ-valerolactone, as well as pluronics, carbonates (e.g., trimethylene carbonate, tetramethylene carbonate, and the like), dioxanones (e.g., 1,4-dioxanone and p-dioxanone), 1,dioxepanones (e.g., 1,4-dioxepan-2-one and 1,5-dioxepan-2-one), and combinations thereof. Polymers formed therefrom include: polylactides; poly(lactic acid); polyglycolides; poly(glycolic acid); poly(trimethylene carbonate); poly(dioxanone); poly(hydroxybutyric acid); poly(hydroxyvaleric acid); poly(lactide-co-(ϵ-caprolactone-)); poly(glycolide-co-(ϵ-caprolactone)); polycarbonates; poly(pseudo amino acids); poly(amino acids); poly(hydroxyalkanoate)s; polyalkylene oxalates; polyoxaesters; polyanhydrides; polyortho esters; and copolymers, block copolymers, homopolymers, blends, and combinations thereof. In certain embodiments, the biocompatible substrate may be formed using a combination of bioabsorbable and non-bioabsorbable polymers.

Examples of non-bioabsorbable materials include polyolefins, such as polyethylene and polypropylene including atactic, isotactic, syndiotactic, and blends thereof; polyethylene glycols; polyethylene oxides; ultra high molecular weight polyethylene; copolymers of polyethylene and polypropylene; polyisobutylene and ethylene-alpha olefin copolymers; fluorinated polyolefins, such as fluoroethylenes, including expanded polytetrafluoroethylene (ePTFE) and condensed polytetrafluoroethylene c(PTFE), fluoropropylenes, fluoroPEGSs, and polytetrafluoroethylene; polyamides, such as nylon and polycaprolactam; polyamines; polyimines; polyesters, such as polyethylene terephthalate and polybutylene terephthalate; aliphatic polyesters; polyethers; polyether-esters, such as polybutester; polytetramethylene ether glycol; 1,4-butanediol; polyurethanes; acrylic polymers and copolymers; modacrylics; vinyl halide polymers and copolymers, such as polyvinyl chloride; polyvinyl alcohols; polyvinyl ethers, such as polyvinyl methyl ether; polyvinylidene halides, such as polyvinylidene fluoride and polyvinylidene chloride; polyacrylonitrile; polyaryletherketones; polyvinyl ketones; polyvinyl aromatics, such as polystyrene; polyvinyl esters, such as polyvinyl acetate; copolymers of vinyl monomers with each other and olefins, such as etheylene-methyl methacrylate copolymers, acrylonitrile-styrene copolymers, ABS resins, and ethylene-vinyl acetate copolymers; alkyd resins; polycarbonates; polyoxymethylenes; polyphosphazine; polyimides; epoxy resins; aramids, rayon; rayon-triacetate; spandex; silicones; and combinations thereof.

### Biocompatible Substrate

A biocompatible substrate comprises at least one surface. A biocompatible substrate may comprise a film made by methods known to those of skill in the art and made from materials disclosed herein. A biocompatible substrate may comprise a woven or nonwoven material. A biocompatible substrate may be formed using any method within the purview of those skilled in the art. Some non-limiting examples include, weaving, knitting, braiding, crocheting, extruding, spraying, casting, molding, electrospinning and combinations thereof. In an aspect, a biocompatible substrate may be a two or three-dimensional surgical mesh which is woven, knitted, braided, or crocheted from at least one first filament to form the substrate. In an aspect, a biocompatible substrate may be a surgical mesh consisting of at least one first filament made of polypropylene or polyethylene terephthalate.

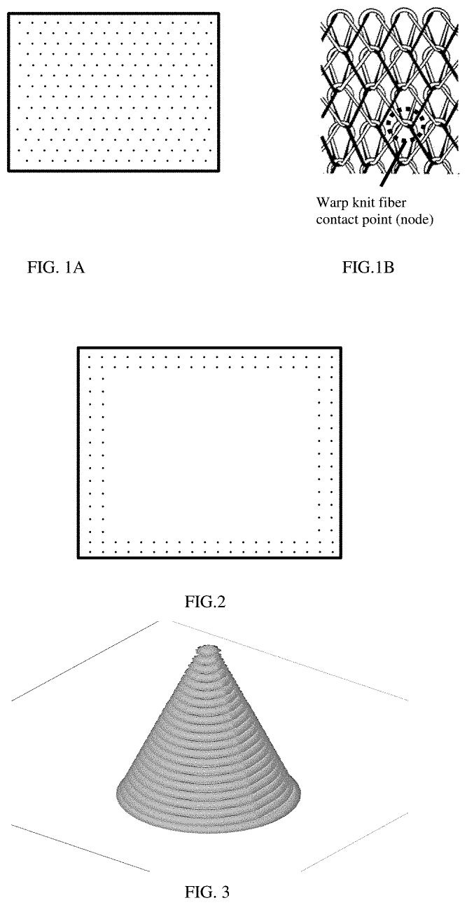

In an aspect, an implantable medical device may be a surgical mesh that is made from a plurality of fibers woven in any suitable manner that allows the fibers to form a substrate and to form contact sites where at least a portion of a fiber contacts a portion of the same or different fiber. FIGS. 1A and 1B show fiber contacting sites onto which are printed fixation elements in accordance with the present disclosure. An implantable medical device may be made on a warp-knitting machine, for example, a tricot or Raschel type.

In an aspect, medical devices disclosed herein may be used for repair of soft tissue and muscle wall defects. Various repair fabrics are known and used for repairing soft tissue and muscle wall defects. Non-limiting examples of implantable fabrics that have been successfully used in soft tissue and muscle wall repair include Soft Mesh®, BARD Mesh®, 3DMAX® Light Mesh and 3DMAX® Mesh and VISILEX®, available from C.R. Bard, and meshes made by Ethicon such as Proceed® Surgical Mesh, by Gore, such as Bio-A Tissue Reinforcement®, and by Medtronic such as Parietex®. Such fabrics may be fabricated from monofilaments (e.g., polypropylene) that are knitted into a mesh having pores or interstices that promote tissue ingrowth and integration with the fabric. Biocompatible substrates contemplated herein include such known implantable fabrics.

In an aspect, an implantable medical device may be configured to fit the shape of the anatomical region of the defect. In some instances, an implantable medical device disclosed herein can be positioned and maintain its position relative to the defect. In an aspect, medical devices are fabricated from a mesh fabric formed into a curved, 3-dimensional shape that fits the anatomical shape of the defect region, such as the breast or inguinal anatomy. Such medical devices have proven useful and have become established in the practice of muscle or tissue wall repair.

### Fixation Elements

Fixation elements, e.g., barbs or nibs, of which a plurality may be positioned on at least a portion of a biocompatible substrate, so that an individual fixation element is sited at an identified site or a fiber contacting site, where at least one portion of a fiber contacts another portion of the same or a different fiber, may be formed from materials disclosed above. A fixation element may be made from any biocompatible, bioabsorbable or non-bioabsorbable material, including those disclosed herein. In an aspect, a biocompatible substrate and the at least one fixation element may be made from the same materials. In an aspect, a biocompatible substrate and the at least one fixation element may be made from different materials. For example, in an aspect, a biocompatible substrate may be formed from at least one filament made from a non-bioabsorbable material, i.e., polypropylene or polyethylene terephthalate, and the fixation elements(e.g., barbs) may be formed from a bioabsorbable material, e.g., a polymeric material comprising polylactic acid and/or polyglycolic acid.

Fixation elements (e.g., barbs or nibs) may comprise a plurality of fixation elements, each fixation element positioned at an identified site and/or a fiber contacting site on a biocompatible substrate. In an aspect, a medical device disclosed herein comprises more than one fixation element, and may comprise a plurality of fixation elements that may be disposed in various arrangements on the biocompatible substrate. In an aspect, one or more fixation elements may be formed on a fiber contacting site, where at least two fibers or two portions of fiber(s) contact each other, by using additive manufacturing devices to print a fixation element at that specific location. In an aspect, one fixation element may be formed on a fiber contacting site, where at least two fibers or two portions of fiber(s) contact each other, by using additive manufacturing devices to print a fixation element at a specific location on a biocompatible substrate. In an aspect, one or more fixation elements may be formed on an identified site by using additive manufacturing devices to print a fixation element at that specific location. A fixation element may be shaped so that the fixation element, e.g., an unattached end (an end opposite from an end attached to the biocompatible substrate) may be uni-directional, multi-directional, symmetrical, non-symmetrical, and combinations thereof.

In an aspect, a fixation element or a plurality of fixation elements may be shaped or may be positioned on the biocompatible substrate so that if moved in one direction, the fixation element(s) do not engage (e.g., do not grip, snag, penetrate or attach to) with a subject's tissue, and when moved in a different direction, the fixation element(s) do engage with (e.g., grip, snag, penetrate or attach to) a subject's tissue. For example, when laparoscopically inserting a biocompatible substrate with at least one fixation element into a subject, the fixation elements may be directionally positioned on the biocompatible substrate so that none of the fixation elements grip, snag, penetrate or attach to a subject's tissue as the medical device is inserted. Once in place within the subject, the medical device is then pulled or tugged in a different direction so that the fixation elements do engage with the subject's tissue.

In an aspect, one or more fixation elements (e.g., barbs or nibs) may extend generally perpendicularly from the surface of a biocompatible substrate. By generally perpendicular, the fixation elements may protrude from the surface of the implant at about 90-degrees. It is envisioned that the fixation elements may protrude from the surface of the implant from about 75 to about 105 degrees.

Tissue-gripping elements, e.g., fixation elements, may be configured in an arrangement on a biocompatible substrate, or each element may have features that help maintain the position of a medical device relative to the subject's defect. The self-affixing arrangement may reduce, if not eliminate, separation, sliding, twisting, folding and/or other movement, as may be desired, between the medical device and adjacent tissue. Such an arrangement may also reduce, if not eliminate, the need for a surgeon or other healthcare provider to suture, staple, tack, or otherwise provisionally anchor the medical device in place pending tissue integration.

In an aspect, a medical device may comprise a plurality of fixation elements protruding from at least one surface of a biocompatible substrate. Fixation elements may protrude from a surface of the body portion of a biocompatible substrate that is configured to engage adjacent tissue. The fixation elements may be configured to penetrate and grip the tissue when the medical device is placed and/or pressed against the tissue. In this manner, fixation elements may be configured to protrude a defined distance from the surface of the biocompatible substrate to penetrate a depth of tissue sufficient to provide the desired amount of grip or attachment. In an aspect, one or more fixation elements may be sited on more than one surface of a medical device.

Fixation elements may be arranged on a biocompatible substrate in any suitable configuration to provide a desired amount of grip, which is apparent to one of skill in the art. For example, and without limitation, fixation elements may be distributed across a biocompatible substrate in a uniform, non-uniform or random array, and/or any suitable combination of arrays. Fixation elements may be distributed across the entire biocompatible substrate or located at one or more select regions of the biocompatible substrate. For example, and without limitation, fixation elements may be located at one or more specific regions adjacent one or more segments of the outer periphery of a biocompatible substrate, and/or one or more specific regions located within the inner region of a biocompatible substrate inwardly away from the outer periphery. Each specific region may include one or more fixation elements arranged in any suitable pattern within the region. One or more of the specific regions may employ the same or different arrangements of fixation elements relative to one or more other specific regions of tissue-gripping elements.

A biocompatible substrate may comprise one or more types of fixation elements, in one region, in differing regions, or having types of fixation elements intermixed on the biocompatible substrate. By types, it is mean that a type comprising a particular characteristic such as the chemical composition of a fixation element, or a physical shape, or a combination of one or more characteristics. For example, a biocompatible substrate may comprise one type of fixation elements comprising one or more fixation elements made from a particular polymer or copolymer, and a second type of one or more fixation elements made from a different polymer or copolymer. A type of fixation element may also comprise one or more fixation elements having a particular shape, such as pyramidal or hooked, and a second or other type(s) having a different shape(s). In an aspect, a type of fixation element may comprise one or more fixation elements made of a particular polymer or copolymer having a particular shape, and another type of fixation elements made of a different polymer or copolymer having the same or a different shape.

In an aspect, fixation elements may be fabricated independently of and mounted to a biocompatible substrate of the medical device. For example, independent fixation elements, e.g., barbs, are manufactured by, and affixed by, a 3-D printing apparatus to one or more identified sites and/or fiber contacting sites of a biocompatible substrate In this manner, fixation elements may be formed from a material that is the same as or different from a biocompatible substrate. For example, and without limitation, fixation elements may be formed of a bioabsorbable material, while the biocompatible substrate may be formed of a non-absorbable material. Alternatively, the biocompatible substrate and the fixation elements may be made of bioabsorbable materials, and such bioabsorbable materials may be the same for the substrate and the fixation elements, or the substrate may be made of bioabsorbable materials different from those of the bioabsorbable materials of the fixation elements. Additionally, among the fixation elements, the fixation elements may have the same or different chemical and/or physical characteristics. Such an arrangement may provide the medical device with temporary tissue-gripping properties during the period of tissue integration, while reducing the amount of foreign material that remains present in a subject's body.

Independent fabrication of fixation elements may also provide flexibility for configuring an implantable device. For example, and without limitation, an implantable medical device may include fixation elements having the same or different configurations and/or arrangements depending on a particular application of the medical device. For example, and without limitation, an implantable medical device may include fixation elements having the same shape, but mounted in different orientations relative to each other on one or more surfaces of a biocompatible substrate. An implantable medical device may include fixation elements with one or more different shapes in one or more regions of the body portion. In this manner, an implantable medical device may be provided with various tissue-gripping characteristics based on the particular orientations and/or shapes of the fixation elements individually and as a whole. Additionally, among the fixation elements, the fixation elements may have the same or different chemical and/or physical characteristics.

In an aspect, any suitable fixation elements' arrangement may be provided on a biocompatible substrate to provide a desired amount of tissue-gripping capability. For example, and without limitation, a single row of fixation elements may be located along one or more specific regions, such as the outer periphery, of a biocompatible substrate. In an aspect, one or more of the specific regions may employ the same or different arrangements of fixation elements relative to one or more other specific regions of fixation elements. The fixation elements may be arranged in a uniform, non-uniform or random array, and/or any suitable combination of arrays. Rather than limited to one or more specific fixation element regions, fixation elements may be distributed across the entire surface or more than one surface of an implantable medical device. Additionally, among the fixation elements, the fixation elements may have the same or different chemical and/or physical characteristics. In an aspect, an implantable medical device may include one or more fixation elements having the same or different fixation element configurations and/or arrangements depending on a particular application of the medical device. For example, and without limitation, an implantable medical device may include fixation elements having the same shape, but mounted in different orientations relative to each other on the biocompatible substrate. An implantable medical device may include fixation elements with one or more different shapes in one or more regions of the body portion. In this manner, the medical device may be provided with various tissue-gripping characteristics based on the particular orientations and/or shapes of the fixation elements individually and as a whole. Additionally, among the fixation elements, the fixation elements may have the same or different chemical and/or physical characteristics.

### Bioactive Agents

Disclosed herein are implantable medical devices comprising fixation elements, and at least one bioactive agent. The at least one bioactive agent may be in a coating on all or a portion of the medical device, and/or may be incorporated into the materials used to form all or a portion of a biological substrate and/or all or a portion of the fixation elements. In an aspect, a biocompatible substrate and/or fixation elements of the medical device can be coated with a bioactive agent. The term “bioactive agent”, as used herein, is used in its broadest sense and includes any substance or mixture of substances that have diagnostic, therapeutic or clinical use. Consequently, bioactive agents may or may not have pharmacological activity per se, e.g., a dye. Alternatively, a bioactive agent could be any agent that provides a therapeutic or prophylactic effect, a compound that effects or participates in tissue growth, cell growth, cell differentiation, or an anti-adhesive compound, a compound that may be able to invoke a biological action such as an immune response, or could play any other role in one or more biological processes. It is envisioned that the bioactive agent may be applied to the substrate and/or fixation elements in any suitable form, e.g., films, powders, liquids, gels, and the like. In an aspect, at least one bioactive agents may be incorporated into the materials used to form a biocompatible substrate and/or one or more tissue-gripping elements. For example, a bioactive agent may be incorporated during the formation of fibers used to weave a biocompatible substrate, or into fibers or compositions used to form tissue-gripping elements, or a bioactive agent may be present in a solution to which a biocompatible substrate and/or fixation elements are exposed so that the bioactive agent is absorbed by or adsorbed to the biocompatible substrate and/or tissue-gripping elements.

### Exemplary Bioactives

Examples of classes of bioactive agents, which may be utilized in accordance with the present disclosure include: anti-adhesives; antimicrobials; analgesics; antipyretics; anesthetics; antiepileptics; antihistamines; anti-inflammatories; cardiovascular drugs; diagnostic agents; sympathomimetics; cholinomimetics; antimuscarinics; antispasmodics; hormones; growth factors; muscle relaxants; adrenergic neuron blockers; antineoplastics; immunogenic agents; immunosuppressants; gastrointestinal drugs; diuretics; steroids; lipids; lipopolysaccharides; polysaccharides; platelet activating drugs; clotting factors; and enzymes. It is also intended that combinations of bioactive agents may be used.

Anti-adhesive agents can be used to prevent adhesions from forming between the mesh and the surrounding tissues opposite the target tissue. In addition, anti-adhesive agents may be used to prevent adhesions from forming between the coated implantable medical device and the packaging material. Some examples of these agents include, but are not limited to hydrophilic polymers such as poly(vinyl pyrrolidone), carboxymethyl cellulose, hyaluronic acid, polyethylene oxide, poly vinyl alcohols, and combinations thereof.

Suitable antimicrobial agents which may be included as a bioactive agent include: triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine; polymyxin, tetracycline; aminoglycosides, such as tobramycin and gentamicin; rifampicin; bacitracin; neomycin; chloramphenicol; miconazole; quinolones such as oxolinic acid, norfloxacin, nalidixic acid, pefloxacin, enoxacin and ciprofloxacin; penicillins such as oxacillin and pipracil, nonoxynol 9, fusidic acid, cephalosporins; and combinations thereof. In addition, antimicrobial proteins and peptides such as bovine lactoferrin and lactoferricin B may be included as a bioactive agent.

Other bioactive agents, which may be included as a bioactive agent include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g., oxybutynin); antitussives; bronchodilators; cardiovascular agents, such as coronary vasodilators and nitroglycerin; alkaloids; analgesics; narcotics such as codeine, dihydrocodeinonc, meperidine, morphine and the like; non-narcotics, such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents, such as hormonal agents, hydrocortisone, prednisolone, prednisone, non-hormonal agents, allopurinol, indomethacin, phenylbutazone and the like; prostaglandins and cytotoxic drugs; chemotherapeutics, estrogens; antibacterials; antibiotics; anti-fungals; anti-virals; anticoagulants; anticonvulsants; antidepressants; antihistamines; and immunological agents.

Other examples of suitable bioactive agents, which may be included in the biocompatible substrate or fixation elements include: viruses and cells; peptides, polypeptides and proteins, as well as analogs, muteins, and active fragments thereof; immunoglobulins; antibodies; cytokines (e.g., lymphokines, monokines, chemokines); blood clotting factors; hemopoietic factors; interleukins (IL-2, IL-3, IL-4, IL-6); interferons (β-IFN, α-IFN and γ-IFN); erythropoietin; nucleases; tumor necrosis factor; colony stimulating factors (e.g., GCSF, GM-CSF, MCSF); insulin; anti-tumor agents and tumor suppressors; blood proteins such as fibrin, thrombin, fibrinogen, synthetic thrombin, synthetic fibrin, synthetic fibrinogen; gonadotropins (e.g., FSH, LH, CG, etc.); hormones and hormone analogs (e.g., growth hormone); vaccines (e.g., tumoral, bacterial and viral antigens); somatostatin; antigens; blood coagulation factors; growth factors (e.g., nerve growth factor, insulin-like growth factor); bone morphogenic proteins; TGF-B; protein inhibitors; protein antagonists; protein agonists; nucleic acids, such as antisense molecules, DNA, RNA, RNAi; oligonucleotides; polynucleotides; and ribozymes.

### Methods of Making Disclosed Medical Devices

A biocompatible substrate, for example, a mesh, can be made by known methods. For example, fibers can be wound to create a warp beam for further processing through weaving or warp knitting into a variety of patterns. One or more warp beams may be knit, for example, using a Raschel or Tricot knitter, to produce a warp knit mesh, or a loom could be used to prepare a woven mesh. A mesh may be a single layer or may be three dimensional, for example, a spacer fabric. In an aspect, multiple fiber types can be combined to create multiphase and/or multimaterial mesh. Knit or woven mesh may be collected from these processes as a continuous mesh fabric. The fabric may be heat treated and/or cut to the final mesh implant size, in either order. In an aspect, a mesh biocompatible substrate may also be cleaned to remove process aids and/or surface contaminants. These known methods can be used to create mesh with a wide variety of mechanical properties, including burst strength and ultimate elongation, and physical properties, such as density and pore size. For some biocompatible substrate implants, density and pore size can impact the mesh biocompatibility, for instance mesh with pore size less than 0.5 mm may be useful as a separation barrier while pore sizes greater than 2 mm may support tissue integration, for instance hernia mesh scaffolds.

A biocompatible substrate such as a nonwoven mesh may be prepared directly. For example, melt blown mesh involves extrusion of a polymer solution or melt through multiple fine diameter orifices into a heated air stream, through which the air attenuates the extrudate to small diameters. These fibers are deposited directly onto a collection belt in a nonwoven pattern as a collection of small fibers. Electrospun mesh may be prepared by injecting a solution through a fine orifice to which a static electric charge is applied. A differential in electric charge between the orifice and collector draws the solution, and this along with solvent evaporation creates nano- and micro-scale fibers. Mesh may be cut to final size and may or may not be heat-treated. Mesh does not typically have a defined macroporous structure, but may be cut to include fenestrations or other holes. In some cases, nonwoven mesh may be added or inserted (i.e. weft insertion) into other knit or woven mesh.

In an aspect, a method of preparing a biocompatible substrate having at least one fixation element located on at least one fiber contacting site may comprise some or all of the following steps. A biocompatible substrate, such as a mesh, is examined to determine the sites of the biocompatible substrate (mesh) at which one portion of a fiber contacts another portion of the same or a different fiber. For example, fiber contacting sites are identified by comparing the initial biocompatible substrate (mesh) structure that has fiber contacting sites to an engineering specification that provides desired or proposed fiber contacting sites, and then fitting the existing sites of the mesh where fibers contact (fiber contacting sites) to the specification's desired locations, thus identifying fiber contacting sites of the biocompatible substrate (mesh) that fall within the specification's proposed locations for fiber contacting sites. The engineering specification may comprise a map with coordinates for locating each fiber contacting site of the biocompatible substrate (mesh) in the 2-D or 3-D dimensional map. The determined locations (coordinates) are then used as fiber contacting sites to which fixation elements can be printed. For example, the desired coordinates (outputs) are entered into a computer program that directs an additive manufacturing printer to print one or more fixation elements at a desired location (coordinates) on the biocompatible substrate.

In general, a method for making a biocompatible mesh comprising fixation elements comprises, 1) Identify sites on a biocompatible substrate that are suitable to function as a fiber contacting site; 2) Map sites (assign coordinates) to create a location index of fiber contacting sites; 3) Compare site locations (coordinates) to an engineering drawing or specification comprising desired fiber site locations for a biocompatible substrate of a medical device; 4) Selecting sites that match the engineering drawing or specification; 5) Output actual coordinates of select fiber crossover sites into a program which drives the printing of fixation elements, and 6) printing one or a plurality of fixation elements on a biocompatible substrate.

A biocompatible substrate without fibers, for example, a film or foam, can be made by known methods. In an aspect, a method of preparing a biocompatible substrate without fibers may comprise some or all of the following steps. A biocompatible substrate, such as a film, is examined to determine (“identify”) the one or more sites of the biocompatible substrate where one or fixation elements are to be printed. For example, identified sites may be selected based on the intended use of the medical device or may be randomly selected, or may be selected based on a pre-determined pattern. An engineering specification may be made of the selected sites and comprise a map with coordinates for locating each identified site of the biocompatible substrate in a 2-D or 3-D dimensional map. The determined locations are then used as identified sites to which fixation elements can be printed. For example, the desired coordinates (outputs) are entered into a computer program that directs an additive manufacturing printer to print one or more fixation elements at a desired location (coordinates) on the biocompatible substrate.

In general, a method for making a biocompatible mesh comprising fixation elements comprises, 1) Identify sites on a biocompatible substrate that are suitable to function as an identified site; 2) Map sites (assign coordinates) to create a location index of identified sites; 3) Compare site locations (coordinates) to an engineering drawing or specification comprising desired identified site locations for a biocompatible substrate of a medical device; 4) Selecting sites that match the engineering drawing or specification; 5) Output actual coordinates of select identified sites into a program which drives the printing of fixation elements, and 6) printing one or a plurality of fixation elements on a biocompatible substrate.

Alternatively, a biocompatible substrate (e.g., mesh) may be supported by a fixture (such as clamps or a frame) to assure the biocompatible substrate (mesh) fiber contacting sites are positioned at the desired locations of a predetermined pattern of fiber contacting sites. Adjustments to fit a predetermined pattern may be made in a 2-dimensional or a 3-dimensional direction.

One or more additive manufacturing printers may be used to form a medical device comprising a biocompatible substrate comprising at least one fixation element. The printers may be of the same type, such as serially using two FDM printers, or may be a FDM printer followed by a SLA printer, or vice versa. In an aspect, one additive manufacturing printer is used to print each fixation element found on a biocompatible substrate. For example, an FDM 3-D printer uses the revised (measured or determined) or existing identified site and/or a fiber contacting site as the location to form at least one fixation element at each desired identified site and/or a fiber contacting site, depending on the desired pattern of fixation elements for the medical device. A printer may use a plurality of sites to print a plurality of fixation elements, generally printing one fixation element on one fiber contacting site, and/or printing one fixation element on one identified site. The present disclosure contemplates fixation element-fiber contacting site arrangements such as printing one fixation element on one fiber contacting site, printing two or more fixation elements on one fiber contacting site, printing one fixation element on two or more fiber contacting sites, and combinations of these arrangements or individual arrangements may be printed on at least one surface of a biocompatible substrate. The present disclosure contemplates fixation element-identified site arrangements such as printing one fixation element on one identified site, printing two or more fixation elements on one identified site, printing one fixation element on two or more identified sites, and combinations of these arrangements or individual arrangements may be printed on at least one surface of a biocompatible substrate.

A fixation element may be printed on each fiber contacting site determined on a medical device comprising fibers, such as a mesh or electrospun article, or may print on only select sites which are distributed across one or more surfaces of the medical device. Fixation elements may be evenly distributed across the entirety of at least one surface of the medical device (meaning that the fixation elements are printed in a pattern, such as on every fiber contacting site or every other fiber contacting site or every third fiber contacting site, etc., in an area of a surface of a medical device) or may be evenly distributed in only certain areas of at least one surface of a medical device, for example, only the periphery of at least one surface of the medical device. Fixation elements may be unevenly distributed (meaning the fixation elements are not printed in a particular pattern) to account for variations in fixation requirements at different points of the medical device. Fixation elements may be located on the top surface of the medical device or on multiple medical device surfaces, for instance the technical face and technical back of the medical device.

A fixation element may be printed on each identified site determined on a medical device not made of fibers, such as a film or a foam, or may print on only select identified sites which are distributed across one or more surfaces of a medical device. Fixation elements may be evenly distributed across the entirety of at least one surface of the medical device (meaning that the fixation elements are printed in a pattern, such as on every identified site or every other identified site or every third identified site, etc., in an area of a surface of a medical device) or may be evenly distributed in only certain areas of at least one surface of a medical device, for example, only the periphery of at least one surface of the medical device. Fixation elements may be unevenly distributed (meaning the fixation elements are not printed in a particular pattern) to account for variations in fixation requirements at different points of the medical device. Fixation elements may be located on the top surface of the medical device or on multiple medical device surfaces, for instance the technical face and technical back of the medical device.

A UV-curable ink may be used to form the 3D printed elements through one of many additive manufacturing processes, for example a UV curable ink may be injected through a syringe-based system to form a fixation element at a fiber contacting site of a biocompatible substrate and cured in place with an appropriate light source. A UV-curable ink may be jetted (jet printed) onto a mesh surface or may be formed separately from the biocompatible surface and later (after curing, for example) the fixation element is attached to the surface by an adhesive, for example a UV curable adhesive. 3D printed (additive manufactured) fixation elements may be produced one at a time, or could be produced several at a time by employing multiple 3D printing heads, or by providing multiple curing sites on a medical device (such as by providing UV light at multiple sites on a medical device to cure UV-curable ink at those locations to form fixation elements).

Fixation elements may be manufactured onto identified sites and/or fiber contacting site so that the biocompatible surface comprises one or more locations comprising a single fixation element per site. For example, the fiber contacting site may constitute a single contact point from two filaments (regardless of whether the two filaments are separate filaments or different locations on the same filament),or a fiber contacting site may comprise multiple filaments contacting, such as in an extended knot, particularly in warp knit mesh, thereby increasing the area of the fiber contacting site. Such larger fiber contact sites provide an increased surface for printing and adhesion of a 3D printed fixation element. In some cases, including woven mesh and small-pore knit mesh, a single 3D printed fixation element may contact more than one fiber contact sites. In the case of nonwoven mesh, where fiber contact sites are distributed throughout the mesh and not localized through the manufacturing process, a single 3D printed fixation element may contact many fiber-contacting sites. Fiber contacting sites are determined and used to locate 3D printed fixation elements. However, supporting the directional stability of one or more fixation elements can be considered. For example, a 3D printed fixation element placed on a single fiber may twist and turn with that fiber, but, in contrast, a fiber contacting site creates a defined plane with multiple fibers defining the orientation so that the fixation element is less likely to twist or to have a changing orientation. The orientation of the fixation element may or may not be related to its function in fixation, for drug delivery, for tissue separation, and other intended use of the element.

### Uses of Disclosed Medical Devices

In an aspect, the disclosure is directed to methods of use of an implantable medical device comprising one or more fixation elements for treatment, repair, reconstruction, and/or augmentation, of one or more anatomical sites, and is suitable for mending defects in, and weaknesses of, soft tissue and muscle walls or other anatomical regions. In an aspect, disclosed methods and medical devices comprise devices for augmenting a subject's body, such as for uses in plastic surgery. The phrase “mending a defect” includes acts of repairing, augmenting, and/or reconstructing a defect and/or a potential defect. In an aspect, an implantable medical device disclosed herein may be used in methods for mending a groin defect including, but not limited to, one or more of an indirect inguinal hernia, a direct inguinal hernia, a femoral hernia and/or other weakness or rupture of the groin anatomy, or for other hernias within a subject's body. It should be understood that a disclosed medical device is not so limited and may be employed in other anatomical procedures, as should be apparent to one of skill in the art. For example, and without limitation, a medical device disclosed herein may be employed for ventral hernias, chest or abdominal wall reconstruction, or large defects, such as those that may occur in obese patients. A disclosed medical device may include one or more features, each independently or in combination, contributing to such uses.

The disclosure comprises an implantable medical device, which includes a biocompatible substrate comprising one or more fixation elements, which may be a repair fabric having a body portion that is configured to cover or extend across the defect opening or weakness when the biocompatible substrate is placed against the defect. A disclosed medical device may be in the form of a patch, although the medical device may employ other configurations as should be apparent to one of skill in the art. A patch may have a planar or non-planar configuration suitable for a particular procedure employed or a particular location in a subject's body.

A disclosed medical device may be used for mending soft tissue and muscle wall defects using various surgical techniques, including open, laparoscopic, hybrid (e.g., Kugel procedure), and robotic techniques. During open procedures, an implantable medical device may be placed through a relatively large incision, for example, an incision made in the abdominal wall and layers of tissue. Then, the defect is filled or covered with the medical device. During laparoscopic and hybrid procedures, the medical device may be collapsed, such as by rolling or folding, into a reduced configuration for entry into a subject, either directly through a comparatively smaller incision or through a laparoscopic cannula that is placed through the incision. The medical device may have particular application with robotic procedures in which placement of the medical device is achieved using surgical robotic tools which may involve passage of the prosthesis through a relatively small cannula (e.g., 8 mm diameter) as compared to a cannula (e.g., 10-12 mm diameter) typically employed for more conventional laparoscopic techniques.

A disclosed device can further comprise a protective covering that will enable the device to be introduced into the body without the fixation element engaging the tissue. Once the device is at the approximate site for its intended use, the protective covering can be removed, thereby exposing the fixation elements. The device can then be manipulated such that the fixation elements engage with the tissue. In an aspect, the protective covering can be a film. In an aspect, the protective film can have a surface that is lubricious. In an aspect, the protective covering can be in a tubular form. In another aspect, the protective covering can be in the form of a film that covers one surface of the device. In an aspect, the device can comprise two protective films that cover more than one surface of the device. The protective covering can comprise a non-absorbable polymer.

### Kits

The present disclosure comprises a kit comprising a medical device disclosed herein, optionally further comprising a protective covering, all contained within a container and optionally, further comprising accessory components including, but not limited to, a needle, sheath, guide wire, cannula, lidocaine, sterile drapes and gloves. The kit may further comprise written instructions for its use.

### Definitions

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, EIZ specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an,” and “the” include plural referents unless the context clearly dictates otherwise. Thus, for example, reference to “a functional group,” “an alkyl,” or “a residue” includes mixtures of two or more such functional groups, alkyls, or residues, and the like.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, when a compound is referred to as a monomer or a compound, it is understood that this is not interpreted as one molecule or one compound. For example, two monomers generally refers to two different monomers, and not two molecules.

As used herein, the terms “optional” or “optionally” means that the subsequently described event or circumstance can or cannot occur, and that the description includes instances where said event or circumstance occurs and instances where it does not.

As used herein, the terms “about,” “approximate,” and “at or about” mean that the amount or value in question can be the exact value designated or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In an aspect, a mammalian subject is a human. A patient refers to a subject afflicted with a disease or disorder. The term “patient” includes human and veterinary subjects. In some aspects of the disclosed methods, the subject has been diagnosed with a need for a treatment comprising providing a medical device disclosed herein.

As used herein, the terms “administering” and “administration” refer to any method of providing a disclosed medical device to a subject.

As used herein, the terms “comprises,” “comprising,” “includes,” “including,” “containing,” “characterized by,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus.

The transitional phrase “consisting of” excludes any element, step, or ingredient not specified in the claim, closing the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” limits the scope of a claim to the specified materials or steps and those that do not materially affect the basic and novel characteristic(s) of the claimed disclosure. A “consisting essentially of” claim occupies a middle ground between closed claims that are written in a “consisting of” format and fully open claims that are drafted in a “comprising” format. Optional additives as defined herein, at a level that is appropriate for such additives, and minor impurities are not excluded from a composition by the term “consisting essentially of”.

When a composition, a process, a structure, or a portion of a composition, a process, or a structure, is described herein using an open-ended term such as “comprising,” unless otherwise stated the description also includes an embodiment that “consists essentially of” or “consists of” the elements of the composition, the process, the structure, or the portion of the composition, the process, or the structure.

The articles “a” and “an” may be employed in connection with various elements and components of compositions, processes or structures described herein. This is merely for convenience and to give a general sense of the compositions, processes or structures. Such a description includes “one or at least one” of the elements or components. Moreover, as used herein, the singular articles also include a description of a plurality of elements or components, unless it is apparent from a specific context that the plural is excluded.

The term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such.

The term “or”, as used herein, is inclusive; that is, the phrase “A or B” means “A, B, or both A and B”. More specifically, a condition “A or B” is satisfied by any one of the following: A is true (or present) and B is false (or not present); A is false (or not present) and B is true (or present); or both A and B are true (or present). Exclusive “or” is designated herein by terms such as “either A or B” and “one of A or B”, for example.

In addition, the ranges set forth herein include their endpoints unless expressly stated otherwise. Further, when an amount, concentration, or other value or parameter is given as a range, one or more preferred ranges or a list of upper preferable values and lower preferable values, this is to be understood as specifically disclosing all ranges formed from any pair of any upper range limit or preferred value and any lower range limit or preferred value, regardless of whether such pairs are separately disclosed. The scope of the disclosure is not limited to the specific values recited when defining a range.

When materials, methods, or machinery are described herein with the term “known to those of skill in the art”, “conventional” or a synonymous word or phrase, the term signifies that materials, methods, and machinery that are conventional at the time of filing the present application are encompassed by this description. Also encompassed are materials, methods, and machinery that are not presently conventional, but that will have become recognized in the art as suitable for a similar purpose.

Unless stated otherwise, all percentages, parts, ratios, and like amounts, are defined by weight.

All patents, patent applications and references included herein are specifically incorporated by reference in their entireties.

It should be understood, of course, that the foregoing relates only to preferred embodiments of the present disclosure and that numerous modifications or alterations may be made therein without departing from the spirit and the scope of the disclosure as set forth in this disclosure.

The present disclosure is further illustrated by the examples contained herein, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present disclosure and/or the scope of the appended claims.

### EXAMPLES

### Example 1

A Hyrel 3D printer (Hyrel, Inc., Atlanta GA) equipped with a direct drive FFF print head with 0.40 mm nozzle was used without modification. A mesh unit measuring 3″×4″ was affixed to the print bed. The mesh was analyzed to determine coordinates for desired printing features (fixation elements), and coordinates input into the printing program. In this example, the mesh was knit from polypropylene monofilament having 100 μm diameter. Knit crossover points (nodes or fiber contacting sites) were spaced at 0.150 inches in an x-direction and 0.248 inches in a y-direction, according to FIG. 1A. FIG. 1B shows a close-up view of a node or knit fiber contacting site.

Polylactide filament (1.75 mm diameter) was printed at 215 ° C. The nozzle was positioned above the input coordinates 2 mm above the surface. 1 mm of filament was extruded from the nozzle and the nozzle was lowered to the mesh surface. The fixation element shape was formed through the extrusion and movement of the printing nozzle away from the print surface. At the end of shape formation, filament extrusion was stopped and the nozzle retracted orthogonally from the fixation element, forming the final fixation element tip.

A total of 40 fixation elements per square inch were printed uniformly across the entire side of the knitted mesh, 3″×4″, with feature points identified as points located at knitting crossover points (nodes or fiber contacting sites) and fixation elements, were printed on alternating horizontal rows.

### Example 2

A knitted mesh measuring 4″×6″ was affixed to a print bed as in Example 1. The mesh was constructed of monofilament polypropylene into a knit structure having nodes (fiber contacting sites) spaced at 0.15 inches in an x-direction and 0.25 inches in a y-direction. The mesh was analyzed to determine coordinates for desired printing features in two rows around the periphery of the mesh, as identified in FIG. 2.

Polycaprolactone filament (1.75 mm diameter) was printed at 185 ° C. using the same printer and nozzle described in Example 1. The nozzle was positioned above the predetermined coordinates 2 mm above the surface. 1 mm of filament was extruded from the nozzle and the nozzle was lowered to the mesh surface. The fixation element shape was formed through the extrusion and movement of the printing nozzle away from the print surface. At the end of shape formation, filament extrusion was stopped and the nozzle retracted parallel to the mesh surface, forming a hook shaped fixation element.

A total of 132 fixation elements were added to the periphery of the knitted mesh, 4″×6″, with feature points identified as points spaced in two rows at 0.25″ spacing. See FIG. 2.

### Example 3

A woven PET mesh tube with a 9 mm diameter was placed on a mandrel. An FFF printer equipped with a rotational axis was used to print fixation elements around the periphery of the mesh tube. The mesh tube pores were less than 0.2 mm.

Lactoflex filament (1.75 mm diameter), a poly(lactide-co-caprolactone-co-trimethylene carbonate), was printed at 195° C. as described in Example 1. Printed fixation elements consisted of a 1.5 mm base and 2 mm height. Three rows of offset nodes (fixation elements) were printed on each end of the tube, with 6.35 mm center to center circumferentially and 3 mm row spacing axially, for a total of 30 printed fixation elements per side of the tube.

After printing, the woven structure was sealed with gelatin for use as a vascular graft.

### Example 4

Polylactide film, 0.1 mm thick×20 cm×20 cm, was affixed to the print bed of an FFF printer equipped with a 0.15 mm nozzle. Polydioxanone filament (1.75 mm diameter) was used to print features on the film surface at a temperature of 160° C. at 0.08 mm layer height. First, the print head was lowered to the film surface. Filament extrusion was initiated and the nozzle moved in a continuous coil pattern to create a hollow cone structure, 2 mm in diameter×2 mm in height, as illustrated in FIG. 3. At the completion of feature formation, filament extrusion was stopped and the nozzle drawn away from the surface of the biocompatible substrate. A total of 80 fixation elements were distributed evenly over the film surface.

What is claimed is:

1. An implantable medical device comprising:
a) biocompatible substrate comprising at least one fiber contacting site formed by a portion of a first fiber contacting a portion of the first fiber or a second fiber or comprising at least one identified site; and
b) one or more fixation elements.

2. The device of claim 1, wherein the one or more fixation elements are positioned or manufactured at the at least one fiber contacting site or at least one identified site, by printing with additive manufacturing methods.

3. The device of claim 2, where a plurality of fixation elements are printed so that more than one fixation element is printed onto one fiber contacting site or one identified site.

4. The device of claim 2, where a plurality of fixation elements are printed so that one fixation element is printed onto one fiber contacting site or one identified site.

5. The device of claim 2, where a plurality of fixation elements are printed so that one fixation element is printed onto more than one fiber contacting site or identified site.

6. The device of claim 1, wherein the biocompatible substrate comprises a film or a foam.

7. The device of claim 1, wherein the biocompatible substrate comprises a woven or nonwoven material.

8. The device of claim 1, wherein the fixation elements are bioresorbable.

9. The device of claim 1, wherein the fixation elements are not bioresorbable.

10. A method for making an implantable medical device, comprising,
1) identifying sites on a biocompatible substrate that are suitable to function as a fiber contacting site or an identified site;
2) mapping sites (assign coordinates) to create a location index of fiber contacting sites or identified sites;
3) comparing site locations (coordinates) to an engineering drawing or specification comprising desired fiber contacting site locations or identified site locations for a biocompatible substrate of a medical device;
4) selecting sites that match the engineering drawing or specification;
5) outputting actual coordinates of select fiber contacting sites or identified sites into a program which drives the printing of fixation elements, wherein the program directs and controls a 3D printing device, and
6) printing one or a plurality of fixation elements on at least one surface of a biocompatible substrate.

11. The device of claim 10, wherein the biocompatible substrate comprises a film or a foam.

12. The device of claim 10, wherein the biocompatible substrate comprises a woven or nonwoven material.

13. The device of claim 10, wherein the fixation elements are bioresorbable.

14. The device of claim 10, wherein the fixation elements are not bioresorbable.

15. A method for the repair or augmentation of a body structure, comprising,
a) administering to an anatomical site of a subject a biocompatible substrate comprising one or more fixation elements.

16. The device of claim 15, wherein the biocompatible substrate comprises a film or a foam.

17. The device of claim 15, wherein the biocompatible substrate comprises a woven or nonwoven material.

18. The device of claim 15, wherein the fixation elements are bioresorbable.

19. The device of claim 15, wherein the fixation elements are not bioresorbable.
